(19) United States
(12) Patent Application Publication
(10) Pub. No.: US 2005/0107806 A1
(43) Pub. Date: May 19, 2005

(54) Hand tool for use in dermabrasion having an abrasive and a vacuum surface

(76) Inventor: Ronald Allan Greenberg, Adelaide (AU)

Correspondence Address: Dr. O. M. (Sam) Zaghmout, 8509 Kernon Ct., Lorton, VA, 22079, US

(21) Appl. No.: 10/985,973

(22) Filed: Nov. 12, 2004

(30) Foreign Application Priority Data
Nov. 14, 2003 (AU) 2003906256

(51) Int. Cl. A61B 17/50

(52) U.S. Cl. 606/131

(57) ABSTRACT

The present invention relates to a hand tool used for dermabrasion, wherein said hand tool includes an abrading surface and a smooth or vacuum surface where the abraded material, typically the skin, can be removed from the surface by suction. This is achieved by providing an abrasive tipped hand-tool including a vacuum aperture wherein the surface surrounding the aperture is of a smooth character enabling the vacuum to be smoothly applied across the skin only when no abrading is taking place.

### CROSS-REFERENCE TO RELATED APPLICATION

This application claims the benefit of Australian Application No. 2003906256, filed 14 Nov. 2003 by the present inventor. The full disclosure of both of these applications is incorporated herein by reference.

### FEDERALLY SPONSORED RESEARCH

Not applicable

### SEQUENCE LISTING OR PROGRAM

Not applicable

### BACKGROUND OF THE INVENTION

The present invention relates to a hand tool used for dermabrasion, wherein said hand tool includes an abrading surface and a smooth or vacuum surface where the abraded material, typically the skin, can be removed from the surface by suction.

Apparatuses for making micro-abrasions, particularly for cosmetic, medical or therapeutic treatment of human tissue such as the removal of scars and other skin blemishes are known. In addition, these apparatus may be used to treat hide or other similar type of materials, which require fine abrasion to remove particular attributes. Typically these apparatus remove scars and other blemishes by removing surface skin layers by the use of abrasive particles or particles.

The particles are generally driven by a pneumatic source, such as a vacuum source. Such an apparatus includes a vacuum pump connected in series by tubes to a recovery container, a handle, and a supply container housing abrasive particles. The handle includes an aperture which when positioned on a surface to be treated prevents any external air for entering the tubes and causes the vacuum pump to draw particles from the supply container through the handle and into the recovery container. Whilst passing through the handle the particles impinge on the surface causing an abrasive action. The particles are then sucked into the recovery container where filters ensure that the particles remain in the recovery container. Typically the particles used are aluminium oxide particles although other particles may be used, if they possess sufficient abrasive properties and are not harmful.

A potential disadvantage of dermabrasion apparatus is that the particles can be lodged in the skin and a substantial amount of aluminium oxide and cells, which have to be properly disposed of, may be left behind on or in the skin. Although no toxic effects have been shown from aluminium oxide left on or in the skin, workers exposed to aluminium often have inflamed lungs. Further, the fine dust may very well damage the cornea of the eyes. For that reason, in using these dermabrasion apparatuses, the operators wear protective glasses and breathing masks. The patients being treated should therefore wear similar protection.

A solution to the aforementioned problem has been to provide for an abrasive tipped hand tool that eliminates the need for a stream of abrasive particles. Such a hand tool, as described in U.S. Pat. No. 6,241,739, provides for a hand tool including a central aperture through which is provided a vacuum. The hand tool is rubbed against the skin causing the dermabrasion. This maintains intimate contact between the abrasive tip and the skin during the treatment process and transports the removed tissue to a collection container. This intimate contact is an essential feature of the patent.

A difficulty with such an arrangement is that the skin elasticity and other properties vary between individuals, the variability also affected by the condition of the persons skin, their age, exposure to sun and so on. Accordingly, it is quite inappropriate for some skin to be suctioned closely to the hand tool since this may lead to uneven abrasion, the skin under suction partially stretched, and under extreme conditions, even damaged.

Further, with a single central aperture, only those portions of the abrasive tip immediately surrounding the aperture carry out the abrasion. Unless the operator ensures that after abrading the skin the central aperture is passed directly over the surface area abraded to collect all of the waste material, residue can remain on the patient.

Another difficulty of the aforementioned hand tool is that the abrasive surface is a flat or planar surface and the operator has to ensure that the hand tool is always positioned against the skin in a particular orientation to achieve an abrading effect.

Yet a further difficulty with known abrasive tipped hand tools is that the size of the abrading particles or particulates on the surface of the tool is typically of the one size. An operator that may to control the amount of abrasion has to therefore replace the abrading tip with another one having more appropriate properties.

It is therefore an object of the present invention to provide a dermabrasion apparatus that overcomes at least some of the abovementioned problems or provides the public with a useful alternative.

This is achieved by providing an abrasive tipped hand-tool including a vacuum aperture wherein the surface surrounding the aperture is of a smooth character enabling the vacuum to be smoothly applied across the skin only when no abrading is taking place.

### SUMMARY OF THE INVENTION

Therefore in one form of the invention there is proposed a device for the removal of the outer surface cells of skin including:
a vacuum source;
a hand tool including an abrasive hemispherical tip for dislodging the cells from the skin being treated, said tip including at least one aperture therethrough and in fluid communication with a bore through said hand tool, said bore connected to said vacuum source wherein the dislodged cells are drawn through at least some of the apertures and through the bore, the surface surrounding the aperture of a smooth character so that during the vacuuming process no abrasion of the skin occurs, the abrasion only occurring when the hand tool is so positioned that there is no vacuuming effect on the skin.

Preferably a recovery container is located in between said bore and said hand tool, said vacuum source adapted to filter out and collect the loose particles suctioned through said hand tool.

In preference said hand tool includes a cylindrical body having a boss, said tip including a head and a peripheral skirt, the skirt adapted to mount over said boss.

Preferably said head is of a hemispherical shape.

In preference a gasket is located in a flute within the boss, said gasket providing for a fluid type seal between said boss and said skirt.

Preferably there is a single central aperture coaxial with the longitudinal axis of said tip. Alternatively there may be several apertures in close proximity to each other.

Alternatively the central aperture is off-axis to the longitudinal axis of the tip.

Preferably said abrasive particles extending across said head vary in their abrading property. Typically the abrading property is determined by the particle size.

In preference said abrasive particles are chosen form the group including but not limited to particles of diamond, aluminium oxide, silicon carbide, silicon oxide or metal nitrides.

In a preferred embodiment said abrasive tip has a mechanically or chemically created roughened surface.

In a further form of the invention there is proposed a method for the removal of the outer surface cells of skin including:
dislodging cells from said skin being treated by the action of an abrasive hemispherical tip of a hand tool, wherein said hand tool includes at least one aperture in fluid communication with a bore which is connected to a vacuum source;
said dislodged cells are drawn through at least some of the apertures and through said bore whereby the surface surrounding the aperture of a smooth character so that during the vacuuming process no abrasion of the skin occurs, the abrasion only occurring when said hand tool is so positioned that there is no vacuuming effect on the skin.

### BRIEF DESCRIPTION OF THE DRAWINGS

The accompanying drawings, which are incorporated in and constitute a part of this specification, illustrate several implementations of the invention and, together with the description, serve to explain the advantages and principles of the invention. In the drawings,

FIG. 1 is a perspective view illustrating a hand tool with an abrasive tip embodying the present invention;

FIG. 2 is a cross-sectional view of the hand-tool in operation;

FIG. 3 is a cross-sectional view of the hand tool embodying the present invention and including particles of different abrading ability;

FIG. 4 is a cross-sectional view of the hand tool embodying the present invention and including an off-axial vacuum aperture;

FIG. 5 is a partial perspective view of a hand tool embodying the present invention and including several in-line vacuum apertures off-axial to the longitudinal axis of the hand tool;

FIG. 6 is a partial perspective view of a hand tool embodying the present invention and including several symmetrically positioned vacuum apertures off-axial to the longitudinal axis of the hand tool, and including differing abrading particles; and

FIG. 7 is a partial perspective view of a hand tool embodying the present invention and including a mesh defining multiple apertures.

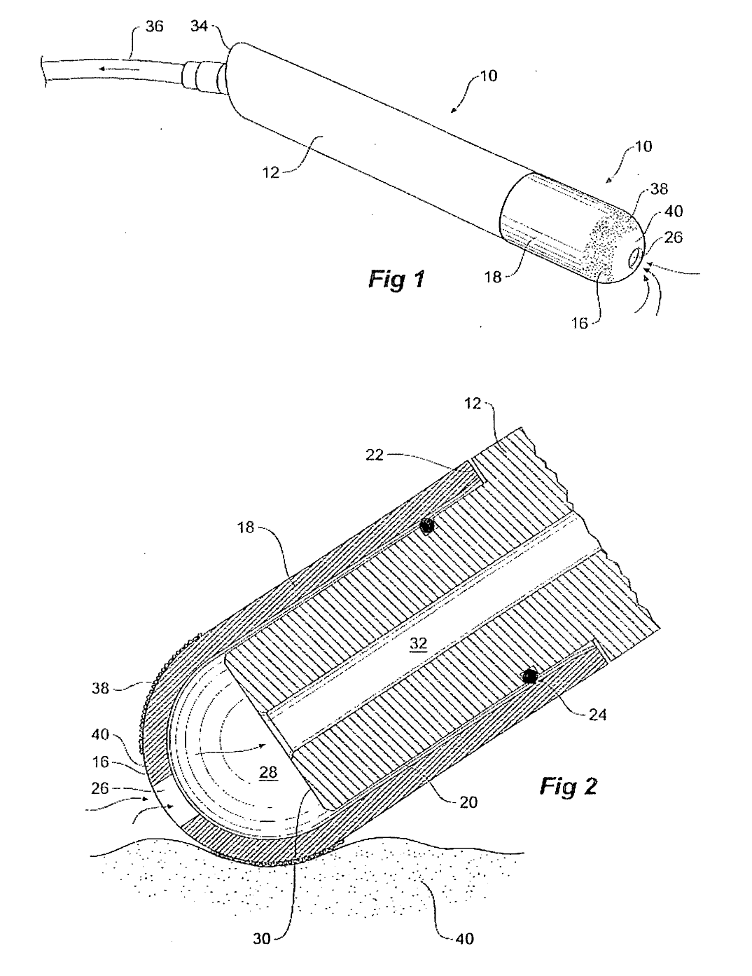

### DESCRIPTION OF THE PREFERRED EMBODIMENTS

The following detailed description of the invention refers to the accompanying drawings. Although the description includes exemplary embodiments, other embodiments are possible, and changes may be made to the embodiments described without departing from the spirit and scope of the invention. Wherever possible, the same reference numbers will be used throughout the drawings and the following description to refer to the same and like parts.

Turning now to the figures in detail there is shown a hand tool 10 including a longitudinal cylindrical body 12 onto which is mounted at one end a tip 14, the tip having a hemi-spherical head 16 and a peripheral skirt 18. The diameter of the body and the skirt are generally the same, the skirt mounting over the body 12, by engaging over the top of boss 20, and abutting against shoulder 22. A gasket ring 24 located generally towards the shoulder 22 provide for an air tight seal between the tip and the body and also acts too provide the resistance necessary to firmly hold the tip mounted on the body.

The head 16 includes a central aperture 26 through which can be drawn air into cavity 28 defined by the head 16 and the end 30 of the boss 20. Extending through the body 12 and the boss 20 is a bore 32 connected at the other end 34 of the body 12 to a tube 36 that by being connected to a vacuum source (not shown) provides for the pneumatic flow.

Away from the aperture 26 and extending circumferentially around the top of the tip are abrading particles 38 that, when placed and rubbed against skin 40, abrade the skin removing dead skin cells. To be able to abrade the skin, the hand tool must be positioned at an angle to the skin surface, as illustrated in FIG. 2, so that the abrading particles are in contact with it. To then vacuum the abraded skin cells, the hand tool is tilted until the aperture is in contact with the skin, much like a vacuum cleaner. The loose skin cells are then drawn up through the aperture 26 of the head 16, into cavity 28, through bore 32, tube 36 and are collected typically in a recovery container (not shown), such containers well known in micro dermabrasion apparatuses.

Typically the head includes a single central aperture with the abrading particles located on the tip starting at around 45-60 degrees from the vertical, with the surface area 40 from the aperture to the abrading particles being a smooth in nature enabling the operator to quickly and cleanly vacuum up the abraded skin.

In contrast with the prior art, when the hand tool is used to abrade the skin, the skin is not sucked up and stretched by the use of vacuum, the vacuuming only achieved when the abrading is no longer taking place.

The skilled addressee would also now understand that by the very nature of the shape of the tool when an operator presses on the skin, the skin is slightly stretched anyway to provide a surface that can be abraded without too much difficulty. This is as a direct result of the tip being of a hemispherical nature, rather than being of a flat configuration.

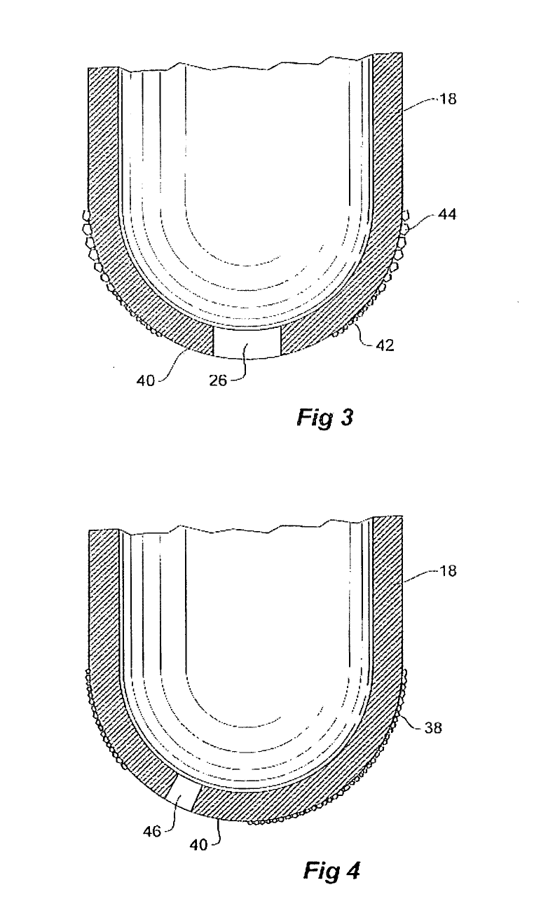

In an alternate embodiment and as illustrated in FIG. 3, there may be different abrading particles on the one tip. Thus, the particles 42 closest to the aperture are of a smaller size causing fine abrading whilst the particles 44 that are further away from the aperture are more abrading. The operator thus not only has the choice of abrading or vacuuming, but also then relative weight of abrading that occurs.

Of course, instead of different abrading, one could equally well have different types of abrading particles, such as organic abrading particles that not only abrade the skin but also leave an organic residue. This is achievable in part because the tip is easily replaceable and can be made a disposable commodity. A user receiving treatment may therefore first receive a deep dermabrasion treatment, followed by an organic treatment by replacing the dermabrading tip with an organic particle tip. The use of the vacuum ensures that any loose particles are always removed.

It is not intended to limit the present invention to the vacuuming aperture being centrally located. For example, the tool may include an off-side aperture 46 as shown in FIG. 4. Such a configuration may suit right or left handed people that would otherwise find it difficult to position the tool correctly. It may also allow a user to be able to effect a much stronger compression on the opposite side of the tip to the aperture in case heavier abrading is required.

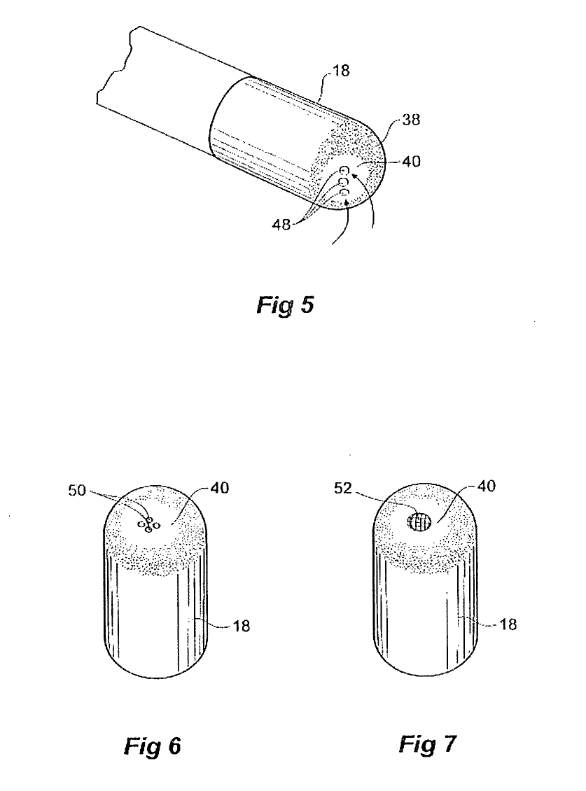

In some cases where the abraded skin is very fine indeed it may be useful to have multiple smaller apertures. Such embodiments are shown in FIGS. 5, 6, and 7, including three in-line apertures 48, four symmetrical apertures 50, and a fine mesh 52 respectively.

Those skilled in the art will immediately appreciate the advantage of the configuration as described above. The abrasive tip can be easily mounted and removed from the body of the hand tool allowing operators to change the tip for different patients. This maintains hygienic standards that are not only expected but also required at times. In fact, it is to be understood that at times the abrasive tips may be disposable type tips and discarded after one use, as may be required in medical applications. Such tips may typically be manufactured from suitable plastics having embedded abrasive particles such as aluminium oxide particles.

Alternatively, the tip may be made from medical grade stainless steel, with the abrasive particles being diamonds. Such a tip may be reusable if it is cleaned in a suitable environment such as an ultrasonic cleaner.

Further advantages and improvements may very well be made to the present invention without deviating from its scope. Although the invention has been shown and described in what is conceived to be the most practical and preferred embodiment, it is recognized that departures may be made therefrom within the scope and spirit of the invention, which is not to be limited to the details disclosed herein but is to be accorded the full scope of the claims so as to embrace any and all equivalent devices and apparatus.

1. A device for the removal of the outer surface cells of skin including:
a vacuum source;
a hand tool including an abrasive hemispherical tip for dislodging the cells from the skin being treated, said tip including at least one aperture therethrough and in fluid communication with a bore through said hand tool, said bore connected to said vacuum source wherein the dislodged cells are drawn through at least some of the apertures and through the bore, the surface surrounding the aperture of a smooth character so that during the vacuuming process no abrasion of the skin occurs, the abrasion only occurring when said hand tool is so positioned that there is no vacuuming effect on the skin.

2. A device for the removal of the outer surface cells of skin as in claim 1 wherein a recovery container is located in between said bore and said hand tool, said vacuum source adapted to filter out and collect the loose particles suctioned through said hand tool.

3. A device for the removal of the outer surface cells of skin as in claim 1 wherein said hand tool includes a cylindrical body having a boss, said tip including a head and a peripheral skirt, said skirt adapted to mount over said boss.

4. A device for the removal of the outer surface cells of skin as in claim 3 wherein said head is of a hemispherical shape.

5. A device for the removal of the outer surface cells of skin as in claim 3 wherein a gasket is located in a flute within said boss, said gasket providing for a fluid type seal between said boss and said skirt.

6. A device for the removal of the outer surface cells of skin as in claim 1 wherein there is a single central aperture coaxial with the longitudinal axis of said tip.

7. A device for the removal of the outer surface cells of skin as in claim 1 wherein said tip includes several apertures in close proximity to each other.

8. A device for the removal of the outer surface cells of skin as in claim 1 wherein said tip includes a central aperture that is off-axis to the longitudinal axis of said tip.

9. A device for the removal of the outer surface cells of skin as in claim 1 wherein said tip includes said abrasive particles extending across the head vary in their abrading property.

10. A device for the removal of the outer surface cells of skin as in claim 1 wherein said abrasive particles are chosen from the group including but not limited to particles of diamond, aluminium oxide, silicon carbide, silicon oxide or metal nitrides.

11. A device for the removal of the outer surface cells of skin as in claim 1 wherein said abrasive tip has a mechanically or chemically created roughened surface.

12. A method for the removal of the outer surface cells of skin including:
dislodging cells from said skin being treated by the action of an abrasive hemi-spherical tip of a hand tool, wherein said hand tool includes at least one aperture in fluid communication with a bore which is connected to a vacuum source;
said dislodged cells are drawn through at least some of the apertures and through said bore whereby the surface surrounding the aperture of a smooth character so that during the vacuuming process no abrasion of the skin occurs, the abrasion only occurring when said hand tool is so positioned that there is no vacuuming effect on the skin.

13. A device for the removal of the outer surface cells of skin substantially as hereinbefore describes with reference to the figures.
